(19) United States
(12) Patent Application Publication
(10) Pub. No.: US 2019/0298457 A1
(43) Pub. Date: Oct. 3, 2019

(54) SYSTEM AND METHOD FOR TRACKING AN INTERVENTIONAL INSTRUMENT WITH FEEDBACK CONCERNING TRACKING RELIABILITY

(71) Applicant: KONINKLIJKE PHILIPS N.V., EINDHOVEN (NL)

(72) Inventor: Shyam BHARAT, ARLINGTON, MA (US)

(21) Appl. No.: 16/341,478

(22) PCT Filed: Nov. 8, 2017
(86) PCT No.: PCT/EP2017/078535

§ 371 Date: Apr. 12, 2019

Related U.S. Application Data
(60) Provisional application No. 62/418,849, filed on Nov. 8, 2016.

(51) Int. Cl. A61B 34/20; A61B 8/08; A61B 8/00; G01S 5/30

(52) U.S. Cl. CPC A61B 34/20; A61B 8/0841; A61B 8/4245; A61B 2017/3413; A61B 2034/2063; A61B 8/58; G01S 5/30

(57) ABSTRACT

A system (100) and method (190) for determining the reliability of an ultrasonic tracking device (102) includes a determination device (132) that is configured to receive signals from the ultrasonic tracking device and determine a quantity of sensors (105) of an interventional device (103) in a field of view of the ultrasonic imaging device. An evaluation device (136) correlates a quantity of the sensors in the field of view with a reliability level for the determined orientation of the interventional device and generates a control signal (142) to a feedback device. The feedback device provides feedback to the user concerning the reliability level for the orientation of the interventional device determined by the ultrasonic tracking device. The feedback may be visual and/or audible feedback.

### BACKGROUND

### Technical Field

This disclosure relates to interventional devices and procedures, and more particularly to systems and methods for tracking interventional devices.

### Description of the Related Art

Real-time tracking of interventional devices for interventional procedures, such as biopsy or therapy, is commonly performed utilizing passive sensors that are mounted on the interventional device. For example, for ultrasonic tracking, a plurality of passive ultrasonic sensors may be coupled to the interventional device and an ultrasonic imaging unit is configured to analyze the signal received by the sensor as the beams of the ultrasonic probe sweep the field of view in order to estimate the position and orientation of the sensors in the field of view.

A certain number of ultrasound sensors are required in the field of view in order for the ultrasonic device to accurately track the orientation of the needle. However, due to the relative positioning of the ultrasound probe and the interventional device during an interventional procedure, there may not be a sufficient quantity of sensors in the field of view for accurate determination of the orientation in real-time navigation and tracking. If the portion of the needle shaft in the ultrasound field of view does not contain enough sensors, the ultrasonic imaging device is not able to accurately track the orientation of the needle. Inaccurate tracking of the needle may cause errors during the performance of the interventional procedure and poses an increased risk of injury to the patient.

Therefore, it would be desirable to provide an ultrasonic system and method for tracking an orientation of an interventional device which provides feedback to the practitioner concerning the reliability of the tracking based on the amount of sensors in the ultrasound field of view in order to avoid tracking errors.

### SUMMARY

In accordance with the present principles, a system for determining the reliability of an ultrasonic tracking device which includes an ultrasound transducer and is configured for tracking an orientation of an interventional device having a plurality of sensors includes a determination device. The determination device is configured to receive signals from the ultrasonic tracking device and determine a quantity of the plurality of sensors in a field of view of the ultrasonic tracking device. An evaluation device includes a data structure that correlates a quantity of the plurality of sensors in the field of view with a reliability level for a determined orientation of the interventional device. The evaluation device is configured to compare the quantity of the plurality of sensors in the field of view determined by the determination device with the data structure and generate a control signal to a feedback device to provide feedback concerning the reliability level for the determined orientation.

In another embodiment, a system for determining the reliability of an ultrasonic tracking device which includes an ultrasound transducer and is configured for tracking an orientation of an interventional device having a plurality of sensors includes a workstation. The workstation includes one or more processors, memory and an interface. The memory includes a determination device that is configured to receive signals from the ultrasonic tracking device and determine a quantity of the plurality of sensors in a field of view of the ultrasonic tracking device. The memory also includes an evaluation device which includes a data structure that correlates a quantity of the plurality of sensors in the field of view with a reliability level for a determined orientation of the interventional device. The evaluation device is configured to compare the quantity of the plurality of sensors in the field of view determined by the determination device with the data structure and generate a control signal to a feedback device to provide feedback concerning the reliability level for the determined orientation.

In another embodiment, a method for determining the reliability of an ultrasonic tracking device that is configured for tracking an orientation of an interventional device having a plurality of sensors, includes the steps of receiving signals from the ultrasonic tracking device and determining a quantity of the plurality of sensors in a field of view of the ultrasonic tracking device. The determined quantity of the plurality of sensors is compared to a data structure that correlates a reliability level for a determined orientation of the interventional device with the quantity of the plurality of sensors in the field of view of the ultrasonic tracking device. Feedback concerning the reliability level for the determined orientation is provided.

These and other objects, features and advantages of the present disclosure will become apparent from the following detailed description of illustrative embodiments thereof, which is to be read in connection with the accompanying drawings.

### BRIEF DESCRIPTION OF DRAWINGS

This disclosure will present in detail the following description of preferred embodiments with reference to the following figures wherein:

FIG. 1 is a block/flow diagram showing a system for tracking an interventional instrument with feedback concerning tracking reliability in accordance with one illustrative embodiment;

FIG. 2 is a block/flow diagram showing an ultrasound imaging device of the system in accordance with one illustrative embodiment;

FIG. 3 is a perspective view of an interventional device having four ultrasound sensors in accordance with one illustrative embodiment;

FIG. 4 is a flow diagram showing a method for tracking an interventional device in accordance with one illustrative embodiment;

FIG. 5 is an image showing a B-scan ultrasound image and a plot of the received signal as a function of the ultrasound beam number where there are two sensors in the field of view;

FIG. 6 is an image showing a B-scan ultrasound image and a plot of the received signal as a function of the ultrasound beam number where there are three sensors in the field of view;

FIG. 7 is an image showing a B-scan ultrasound image and a plot of the received signal as a function of the ultrasound beam number where there are four sensors in the field of view; and

FIG. 8 is a flow diagram showing a method for tracking an interventional instrument with feedback concerning tracking reliability in accordance with one illustrative embodiment.

### DETAILED DESCRIPTION OF EMBODIMENTS

In accordance with the present principles, a system for determining the reliability of an ultrasonic tracking device is provided. The system includes a determination device which determines the quantity of sensors of an interventional device in the field of view of the tracking device. An evaluation device determines a reliability level for the orientation of the interventional device determined by the ultrasonic tracking device based on the number of sensors in the field of view. The system provides clear and easily identifiable feedback, such as visual and/or audible feedback, to the user to inform them whether the determined orientation is reliable. The system provides an efficient and effective modality to help a practitioner avoid mistakes during the performance of the interventional procedure due to inaccurate tracking by the ultrasonic tracking device.

The system provides a quality control concerning the tracking determined by an ultrasonic tracking system. The system may also provide feedback to guide the practitioner to reposition the ultrasound probe for improved reliability of the determined orientation.

It should be understood that the present invention will be described in terms of medical tracking systems. However, the teachings of the present invention are much broader and in some embodiments, the present principles are employed in quantitatively evaluating complex biological or mechanical systems. Furthermore, the present principles are applicable to internal evaluation procedures of biological systems in all areas of the body such as the lungs, liver, brain, uterus, gastro-intestinal tract, excretory organs, blood vessels, and any other solid organ tissue, tumor tissue and homogenously or heterogeneously enhancing structures of the body. The elements depicted in the Figs. may be implemented in various combinations of hardware and software and provide functions which may be combined in a single element or multiple elements.

The functions of the various elements shown in the Figs. can be provided through the use of dedicated hardware as well as hardware capable of executing software in association with appropriate software. When provided by a processor, the functions can be provided by a single dedicated processor, by a single shared processor, or by a plurality of individual processors, some of which can be shared. Moreover, explicit use of the term “processor” or “controller” should not be construed to refer exclusively to hardware capable of executing software, and can implicitly include, without limitation, digital signal processor (“DSP”) hardware, read-only memory (“ROM”) for storing software, random access memory (“RAM”), non-volatile storage, etc.

Moreover, all statements herein reciting principles, aspects, and embodiments of the invention, as well as specific examples thereof, are intended to encompass both structural and functional equivalents thereof. Additionally, it is intended that such equivalents include both currently known equivalents as well as equivalents developed in the future (i.e., any elements developed that perform the same function, regardless of structure). Similarly, it will be appreciated that various processes may be substantially represented in computer readable storage media and so executed by a computer or processor, whether or not such computer or processor is explicitly shown.

Furthermore, embodiments of the present invention can take the form of a computer program product accessible from a computer-usable or computer-readable storage medium providing program code for use by or in connection with a computer or any instruction execution system. For the purposes of this description, a computer-usable or computer readable storage medium can be any apparatus that may include, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium. Examples of a computer-readable medium include a semiconductor or solid state memory, magnetic tape, a removable computer diskette, a random access memory (RAM), a read-only memory (ROM), a rigid magnetic disk and an optical disk. Current examples of optical disks include compact disk-read only memory (CD-ROM), compact disk-read/write (CD-R/W), Blu-Ray™ and DVD.

In accordance with the present principles, a system for tracking an interventional instrument with feedback concerning the tracking reliability is provided. While the system may be utilized in connection with numerous tracking modalities utilizing passive sensors, in a preferred embodiment described herein, the system is used for an ultrasonic tracking system.

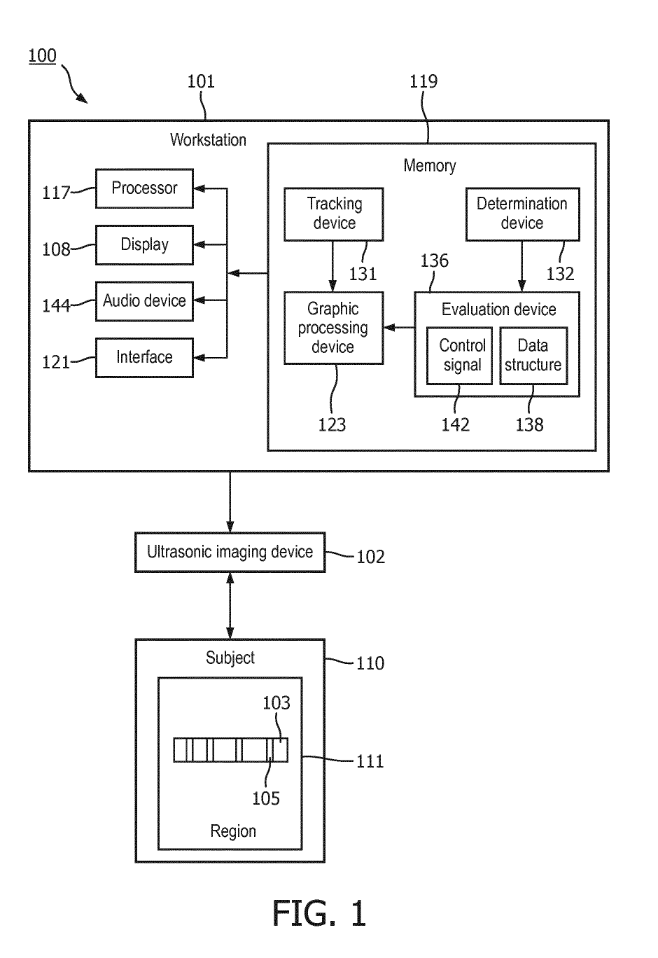

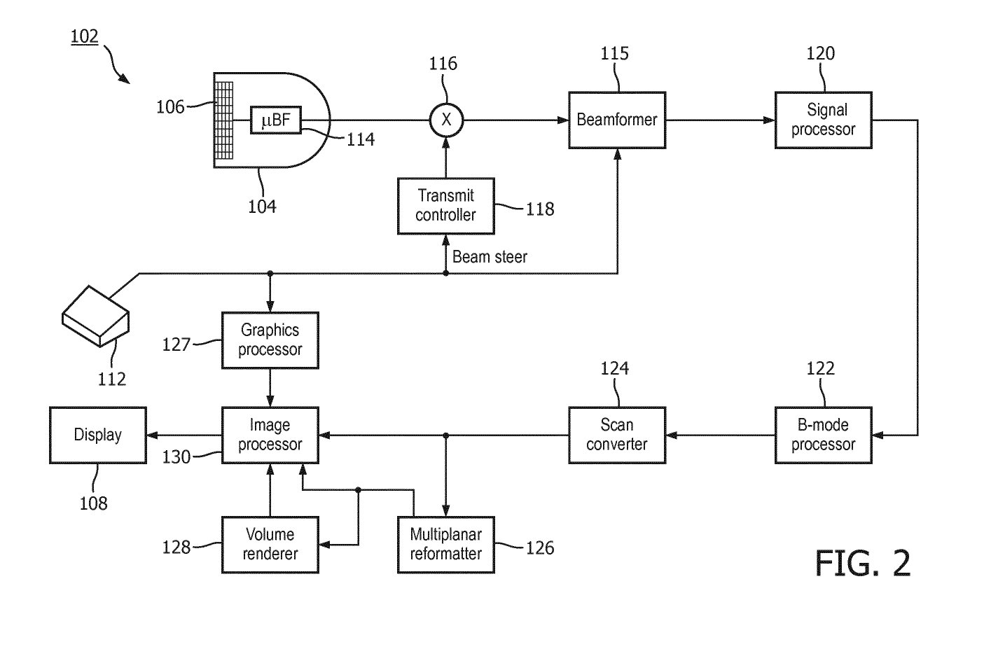

Referring now to the drawings in which like numerals represent the same or similar elements and initially to FIGS. 1-2, a system 100 includes an ultrasonic imaging device 102. The system further includes an interventional device 103 which has a plurality of sensors 105 mounted thereon for performance of an interventional procedure on a region 111 of a subject 110. The sensors may be composed of PZT, PVDF, copolymer or other piezoelectric material or other materials known in the art. In a preferred embodiment, the interventional device 103 is a needle 107. However, in other embodiments, the interventional device 103 may include a catheter, a guidewire, a probe, an endoscope, a robot, an electrode, a filter device, a balloon device or other medical components, etc.

As shown in FIG. 2, the ultrasonic imaging device 102 includes a transducer device or probe 104 having a transducer array 106 for transmitting ultrasonic waves and receiving echo information from the sensors 105 of the interventional device 103. The transducer array 106 may be configured as, e.g., linear arrays or phased arrays, and can include piezoelectric elements or capacitive micromachined ultrasonic transducers (CMUT) elements. The transducer array 106, for example, can include a two dimensional array of transducer elements capable of scanning in both elevation and azimuth dimensions for 2D and/or 3D imaging. The ultrasonic imaging device 102 is preferably a radio frequency (“RF”) ultrasonic imaging device. The ultrasonic imaging device 102 may include a non-handheld probe holder or the probe 104 may be configured for being handheld.

The transducer array 106 is coupled to a microbeamformer 114 integrated within the probe 104, which controls transmission and reception of signals by the transducer elements in the array. The microbeamformer 114 may be coupled to a transmit/receive (T/R) switch 116, which switches between transmission and reception and protects a main beamformer 115 from high energy transmit signals. In some embodiments, the T/R switch 116 and other elements in the system can be included in the transducer probe rather than in a separate ultrasound system base. The transmission of ultrasonic beams from the transducer array 106 under control of the microbeamformer 114 is directed by a transmit controller 118 coupled to the T/R switch 116 and the beamformer 115, which may receive input from the user's operation of a user interface or control panel 112.

One function controlled by the transmit controller 118 is the direction in which beams are steered. Beams may be steered straight ahead from (orthogonal to) the transducer array 106, or at different angles for a wider field of view. The partially beamformed signals produced by the microbeamformer 114 are coupled to a main beamformer 115 where partially beamformed signals from individual patches of transducer elements are combined into a fully beamformed signal.

The beamformed signals are coupled to a signal processor 120. The signal processor 120 may be configured to process the received echo signals in various ways, such as bandpass filtering, decimation, I and Q component separation, and harmonic signal separation. The signal processor 120 may also perform additional signal enhancement such as speckle reduction, signal compounding, and noise elimination. The processed signals are coupled to a B mode processor 122, which can employ amplitude detection for the imaging of structures in the body.

The signals produced by the B mode processor are coupled to a scan converter 124 and a multiplanar reformatter 126. The scan converter 124 arranges the echo signals in the spatial relationship from which they were received in a desired image format. For instance, the scan converter 124 may arrange the echo signal into a two dimensional (2D) sector-shaped format, or a pyramidal three dimensional (3D) image. The multiplanar reformatter 126 can convert echoes which are received from points in a common plane in a volumetric region of the body into an ultrasonic image of that plane, as described in U.S. Pat. No. 6,443,896 (Detmer), which is incorporated herein by reference in its entirety. A volume renderer 128 converts the echo signals of a 3D data set into a projected 3D image as viewed from a given reference point, e.g., as described in U.S. Pat. No. 6,530,885 (Entrekin et al.), which is incorporated herein by reference in its entirety. The 2D or 3D images are coupled from the scan converter 124, multiplanar reformatter 126, and volume renderer 128 to an image processor 130 for further enhancement, buffering and temporary storage for display on an image display 108. A graphics processor 127 is configured to generate graphic overlays for display with the ultrasound images.

In a preferred embodiment, the system 100 may also include a workstation 101 from which the procedure is supervised and/or managed. The workstation preferably includes one or more processors 117, memory 119 for storing programs and applications and a display 108 which permits a user to view images and interact with the workstation. The display 108 of the workstation may be separate or combined with the image display of the ultrasonic imaging device 102.

The system 100 may further include an interface 121 to permit a user to interact with the system and its components and functions. The interface may include a keyboard, mouse, a joystick, a haptic device, or any other peripheral or control to permit user feedback from and interaction with the system. In certain embodiments, the interface 121 of the system may be combined with the interface or control panel 112 of the ultrasonic imaging device 102.

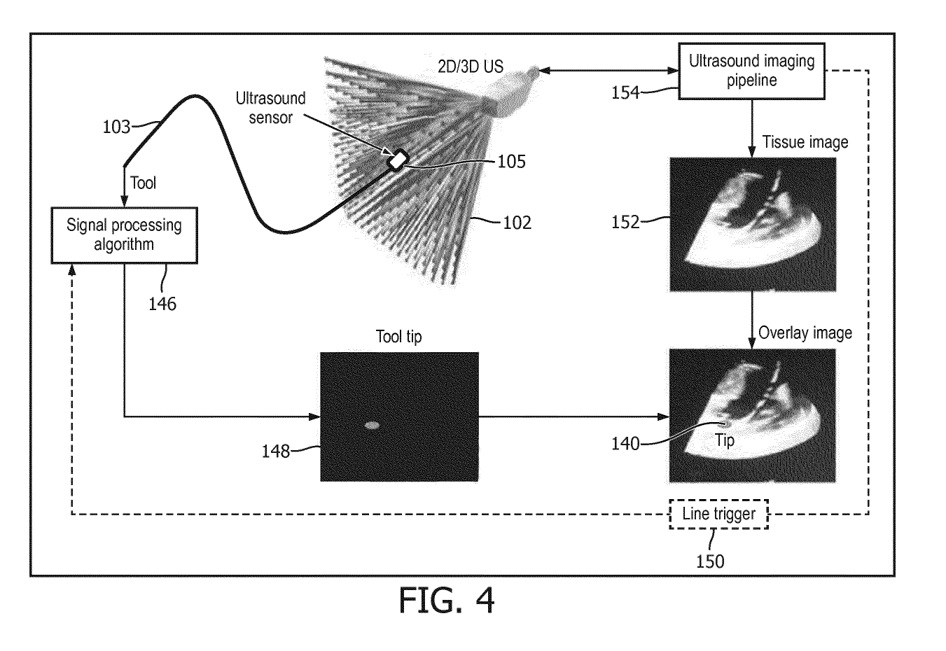

A tracking device 131 is configured to receive signals from the ultrasonic imaging device 102 as the beams of the ultrasound probe 104 sweep the field of view 125 and determine the position and orientation of the ultrasonic sensors 105 on the interventional device 103. The ultrasonic imaging device receives the signals and generates an image 152 by processing the signals through the various components of the ultrasound imaging pipeline 154 as previously described. An illustrative embodiment of the procedure performed by the tracking device 131 is shown in FIG. 4.

In the embodiment shown in FIG. 2, the tracking device is a software-based implementation stored in the memory 119 of the system. The tracking device 131 is configured to perform a signal processing algorithm 146 on the signals received from the ultrasonic imaging device to estimate the position of the sensor. For example, the tracking device 131 may be configured to estimate the position of the sensors 105 by time-of-flight measurements which provide the axial/radial distance of the sensors 105 to the transducer array 106. The tracking device 131 is also configured to analyze the amplitude measurements and knowledge of the beam firing sequence to provide the lateral/angular position of the sensors 105. The tracking device 131 may utilize frame and line trigger signals 150 concerning the beams of energy emitted by the ultrasound transducer 106 in order to determine the location and orientation of the interventional device 103 in an image of the tool which includes the sensor 105, such as a tool tip image 148.

A graphics processing device 123 may be configure to receive the position and orientation information from the tracking device 131 and generate an overlay 140 on the ultrasound image 152 shown on the display 108 representing the determined position and orientation of the sensors 105 on the interventional device 103. The ultrasonic imaging device which incorporates a tracking device 131 may be referred to as an ultrasonic tracking device.

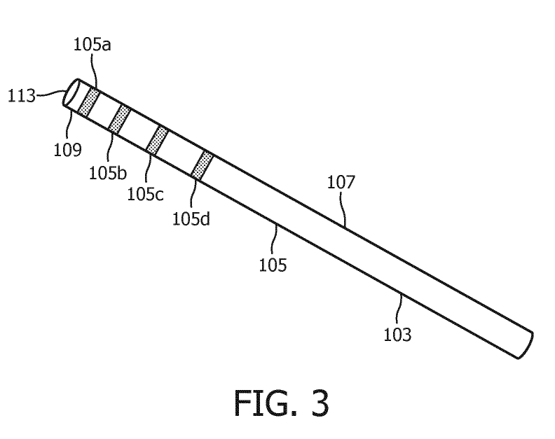

As shown in FIG. 3, the interventional device 103 may have a plurality of ultrasound sensors 105 that are arranged in a characteristic pattern. In one embodiment, each of the ultrasound sensors 105 are spaced apart in a predetermined, unequal manner. The needle 107 shown in FIG. 3 has four sensors. The first sensor 105a is a known distance 109 from the tip 113 of the needle. The first 105a and second 105b sensors, second and third 105c sensors, and third and fourth 105d sensors are all spaced apart in an unequal, known manner as well.

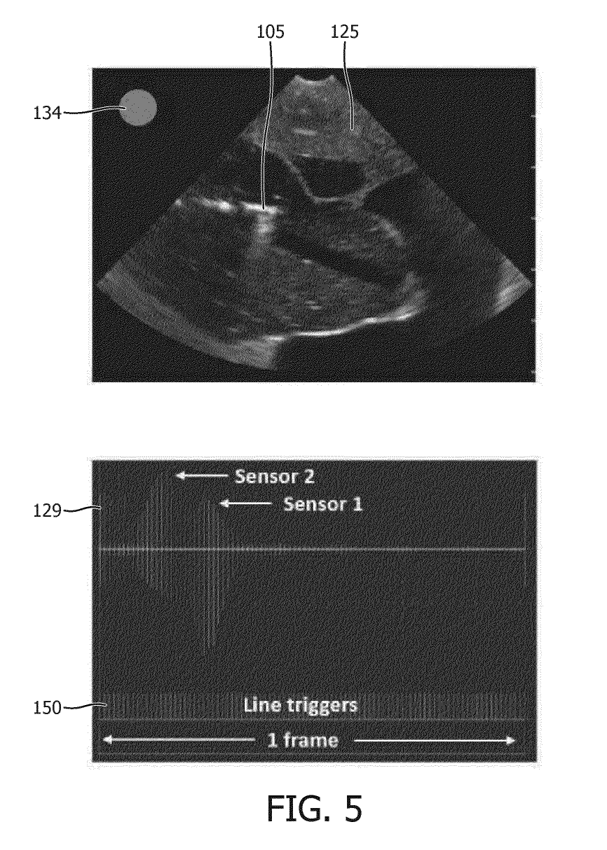

The system 100 further includes a determination device 132. The determination device 132 is configured to receive the ultrasound signals from the signal processor 120 and analyze the signals to determine the quantity of sensors 105 on the interventional device 103 in the field of view. For example, as shown in FIG. 5, the determination device 132 may be configured to receive the signal stream representing the ultrasound echoes received from the ultrasound transducer 106 and based on timing information from the frame and line triggers of the transducer array 106, the determination device 132 may plot the signals captured on the signal trace as a function of the beam/line number for any given frame. The signal stream may be received by the determination device 132 directly from the signal processor 120. However, in other embodiments, the signal stream is received prior to processing of the signal by the signal processor 120 or further downstream of the signal processor in the ultrasonic imaging device 102.

FIGS. 5-7 show signals traced as a function of the beam/line number by the determination device 132. The signals demonstrate a falling and rising signal pattern which corresponds to acoustic pulses received by the sensors 105. The index of a peak signal 129 is indicative of the location of a sensor 105. Therefore, in order to successfully detect the peak sensor signal 129 and the sensor location, both the rising and falling portions of the signal should preferably lie within the ultrasound field of view.

The determination device 132 is configured to analyze the plot of the signals captured on the signal trace as a function of the beam/line number for any given frame and determine which sensors are in the field of view and the direction that the interventional device is moving. The known characteristic spacing of the sensors 105 of the interventional device may be provided to the determination device 132 which allows the determination device to identify each sensor based upon the distance between the sensors sensed by the ultrasonic imaging system and to determine the direction of the needle tip.

The determination device 132 is also configured to detect the peaks 129 in the signal stream which indicate the location of a sensor 105 and determine the number of sensors that are in the field of view. For example, in one embodiment, the determination device 132 utilizes optical recognition to identify a peak 129 in the signal stream using existing optical recognition methods known in the art. The determination device 132 is configured to send a determination concerning the quantity of sensors that are in the field of view to an evaluation device 136.

The evaluation device 136 includes at least one data structure 138, such as a table, which correlates a quantity of sensors in a field of view of the ultrasonic imaging device 102 with an associated reliability/confidence level for the accuracy of the tracking. For example, the orientation of a needle 107 may not be accurately determined with one sensor. In embodiments where the interventional device 103 includes four sensors, accuracy of the determined orientation is greatest when all four sensors are in the field of view of the ultrasound images.

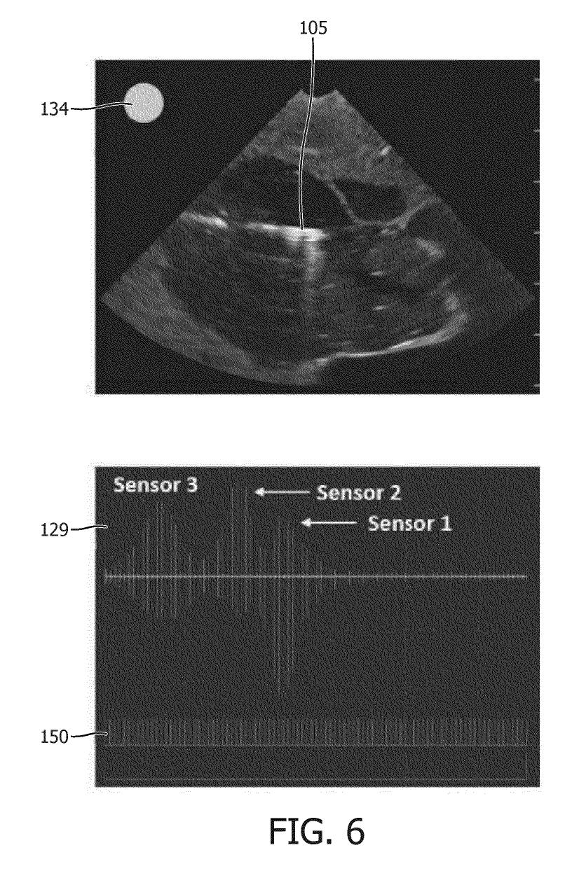

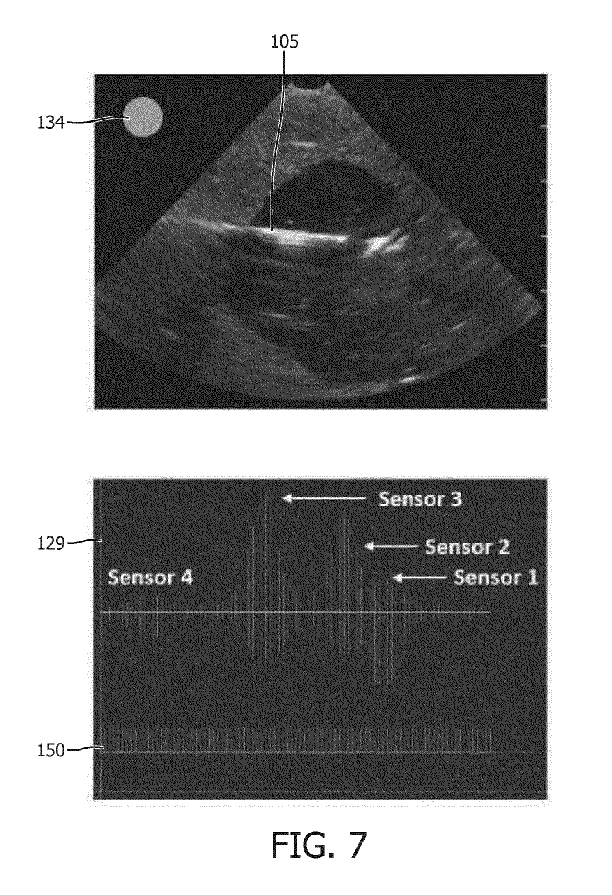

In the embodiment shown in FIG. 5, the signal received from the ultrasonic tracking device which is plotted as a function of the ultrasound beam number reveals two peaks 129 which are determined by the determination device 132 to be indicative of sensors 1 and 2. In the embodiment shown in FIG. 6, the signal received from the ultrasound imaging device 102 reveals three peaks 129 which are determined by the determination device 132 to be indicative of sensors 1-3. In the embodiment shown in FIG. 7, the signal received from the ultrasound imaging device 102 reveals four peaks which are determined by the determination device 132 to be indicative of sensors 1-4.

The evaluation device 136 is configured to review the quantity of sensors determined by the determination device 132 and review the associated reliability/confidence level of the determined orientation stored in the data structure 138. The evaluation device 136 is configured to generate a control signal 142 for providing feedback based on the confidence level associated with the determined quantity of sensors from the data structure 138. For example, one sensor in the field of view is generally insufficient to determine the orientation of the interventional device 105. Therefore, when the determination device 132 determines that only one sensor is in the field of view, the evaluation device is configured to generate a control signal 142 to a feedback modality to provide an alert to the user that the orientation of the sensors cannot be determined. For example, this feedback may be in the form of a predetermined graphical image or message 134, an audible warning, haptic feedback or other methods known in the art.

In the embodiment shown in FIG. 5, the signal trace indicates that two sensors are in the field of view. While the orientation of the interventional device 103 is capable of being determined from two sensors in the field of view, the determined orientation is very sensitive to small errors in the estimated positions of each sensor. Therefore, the determined orientation may be unreliable when there is only two sensors in the field of view. In one embodiment, the system 100 may be configured to send the control signal 142 generated by the evaluation device 136 to a graphic processing device 123. As shown in FIG. 5, the graphic processor may be configured to display a graphical image 134, such as a red circle adjacent the B-mode ultrasound images indicating that the determined orientation of the interventional device is not reliable.

A determination of the orientation of an interventional device 103 having four sensors is generally less sensitive to small variations in the determined sensor positions when there are three sensors 105 in the field of view. Therefore, the data structure 138 may indicate that the reliability of the determined orientation is acceptable if there are three sensors in the field of view. The evaluation device 136 is configured to generate a control signal 142 to a feedback device indicating that the reliability level of the determined orientation is acceptable. The evaluation device 136 may be configured to send the control signal 142 to a graphic processing device 123. As shown in FIG. 6, the graphic processor may be configured to display a yellow circle adjacent the B-mode ultrasound images indicating that the reliability of the determined orientation of the interventional device is acceptable.

The determination of the orientation of an interventional device when there are four sensors 105 in the field of view provides the most robust and accurate orientation estimation in instances where there are four sensors on an interventional device. Therefore, the reliability of the determined orientation may be highest if there are four sensors in the field of view. In this instance, the evaluation device 136 may be configured to generate a control signal 142 to a feedback device indicating that the confidence level of the determined orientation is high. The evaluation device 136 may be configured to send the generated control signal 142 to a graphic processing device 123. As shown in FIG. 7, the graphic processing device 123 may be configured to display a green circle adjacent the B-mode ultrasound images indicating that the determined orientation of the interventional device is most reliable.

While the control signal 142 that is shown in FIGS. 5-7 generates a graphical image 134 having a color corresponding to the reliability of the determined orientation, in other embodiments different graphical images or messages may be utilized to provide feedback to the user concerning the reliability of the determined orientation. Additionally, in other embodiments, the control signal 142 may be sent to an audio device 144 that is configured to emit audible messages to indicate the reliability of the determined orientation. For example, in one embodiment, the audio device 144 may emit periodic audible warning signals when the determined orientation is not reliable or cannot be made. In certain embodiments, the system 100 may be configured to simultaneously provide audio feedback from the audio device 144 as well as visual feedback from the graphics processor 123.

Furthermore, the data structure 138 may have various different confidence/reliability levels associated with the determined quantity of sensors based on the configuration of the interventional device and the ultrasound sensors or other factors.

In a preferred embodiment, the evaluation device 136 is configured to receive the determined number of sensors and their orientation from the determination device 132 and determine a direction that the ultrasound probe 104 should be moved in order to provide a more reliable determination of the orientation. In one embodiment, the evaluation device 136 is configured to generate a control signal 142 to the graphic processor 123 to generate a graphic, such as an arrow, on the display 108 indicating the direction that the user must reposition the probe 104 in order to provide a more accurate determination of the orientation of the interventional device 103.

While FIG. 1 shows various devices including the tracking device 131, graphic processing device 123, determination device 132 and evaluation device 136 stored in the memory 119 as software-based implementations, in other embodiments these devices may be implemented as hardware or combinations of hardware and software.

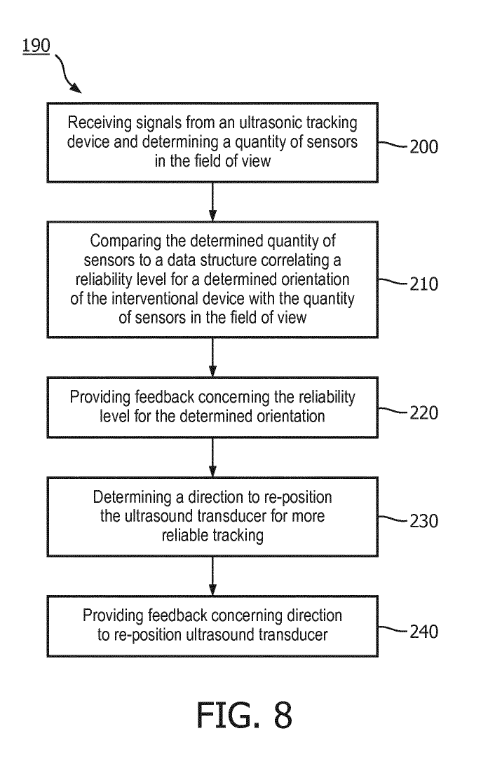

Referring to FIG. 8, a method 190 for determining the reliability of an ultrasonic tracking device is provided. The ultrasonic tracking device is configured for tracking an orientation of an interventional device which has a plurality of sensors. In block 200, signals from the ultrasonic tracking device are received and a quantity of the plurality of sensors in a field of view of the ultrasonic tracking device are determined. The determination of the quantity of sensors in a field of view may be determined by analyzing a signal stream plotted as a function of a beam/line number for a given frame and determining the number of peaks.

In block 210, the determined quantity of sensors is compared to a data structure that correlates a reliability level for a determined orientation of the interventional device with the quantity of the plurality of sensors in the field of view of the ultrasonic tracking device. In block 220, feedback concerning the reliability level for the determined orientation is provided. As previously discussed in detail with respect to the system, the feedback may be visual feedback such as a predetermined graphic. For example, the graphic may be the colored geometric symbol that is displayed adjacent to a B-mode ultrasound image of the interventional device. Alternatively, the feedback may be an audible signal, such as an alarm.

In block 230, the method comprises the further step of determining a direction to re-position an ultrasound transducer to provide a more reliable determination of the determined orientation based on the determined quantity of the plurality of sensors in the field of view and an orientation of the determination device. In block 240, visual feedback such as a graphic is generated on a display indicating the direction to re-position the ultrasound transducer in order to provide improved reliability for the orientation determination.

It is noted that modifications and variations can be made by persons skilled in the art in light of the above teachings. It is therefore to be understood that changes may be made in the particular embodiments of the disclosure disclosed which are within the scope of the embodiments disclosed herein as outlined by the appended claims.

In interpreting the appended claims, it should be understood that:
a) the word “comprising” does not exclude the presence of other elements or acts than those listed in a given claim;
b) the word “a” or “an” preceding an element does not exclude the presence of a plurality of such elements;
c) any reference signs in the claims do not limit their scope;
d) several “means” may be represented by the same item or hardware or software implemented structure or function; and
e) no specific sequence of acts is intended to be required unless specifically indicated.

Having described preferred embodiments the system and method for tracking an interventional instrument with feedback concerning tracking reliability and method (which are intended to be illustrative and not limiting), it is noted that modifications and variations can be made by persons skilled in the art in light of the above teachings. It is therefore to be understood that changes may be made in the particular embodiments of the disclosure disclosed which are within the scope of the embodiments disclosed herein as outlined by the appended claims. Having thus described the details and particularity required by the patent laws, what is claimed and desired protected by Letters Patent is set forth in the appended claims.

1. A system for determining the reliability of an ultrasonic tracking device which includes an ultrasound transducer and is configured for tracking an orientation of an interventional device having a plurality of sensors, comprising:
a determination device configured to receive signals from the ultrasonic tracking device and determine a quantity of the plurality of sensors in a field of view of the ultrasonic tracking device; and
an evaluation device which includes a data structure that correlates a quantity of the plurality of sensors in the field of view with a reliability level for a determined orientation of the interventional device;
wherein said evaluation device is configured to compare the quantity of the plurality of sensors in the field of view determined by the determination device with the data structure and generate a control signal to a feedback device to provide feedback concerning the reliability level for the determined orientation.

2. The system as recited in claim 1, wherein the determination device is configured to plot the signals from the ultrasonic tracking device as a function of a beam or line number for any given frame.

3. The system as recited in claim 2, wherein the signals comprise a signal stream received from a signal processor of the ultrasonic tracking device.

4. The system as recited in claim 1, wherein:
the control signal generated by the evaluation device is sent to a graphic processing device; and
the graphic processing device is configured to generate a predetermined graphic on a display indicative of the reliability level for the determined orientation.

5. The system as recited in claim 4, wherein the predetermined graphic comprises a geometric object having a specific color that indicates the reliability level.

6. The system as recited in claim 5, wherein the graphic processing device is configured to generate the geometric object on a display adjacent to a real-time ultrasound image of the interventional device acquired by the ultrasonic tracking device.

7. The system as recited in claim 1, wherein each of the plurality of the sensors of the interventional device are arranged in a predetermined pattern.

8. The system as recited in claim 1, wherein the interventional device has at least four sensors and the reliability level for the determined orientation of the interventional device comprises:
a high level when the determination device determines that there are four or more sensors in the field of view;
an acceptable level when the determination device determines that there are three sensors in the field of view; and
a low level when the determination device determines that there are two sensors in the field of view.

9. The system as recited in claim 1, wherein the control signal generated by the evaluation device is sent to an audio device and the audio device is configured to generate a predetermined audible message indicative of the reliability level.

10. The system as recited in claim 1, wherein:
the evaluation device is configured to receive the determined quantity of the plurality of sensors in the field of view from the determination device and an orientation of the determination device and determine a direction to re-position the ultrasound transducer to provide a more reliable determination of the determined orientation; and
the evaluation device is configured to generate a second control signal to a graphic processing device to generate a graphic on a display indicating the direction to re-position the ultrasound transducer.

11. A system for determining the reliability of an ultrasonic tracking device which includes an ultrasound transducer and is configured for tracking an orientation of an interventional device having a plurality of sensors, comprising:
a workstation including:
one or more processors, memory and an interface, wherein said memory includes:
a determination device configured to receive signals from the ultrasonic tracking device and determine a quantity of the plurality of sensors in a field of view of the ultrasonic tracking device; and
an evaluation device which includes a data structure that correlates a quantity of the plurality of sensors in the field of view with a reliability level for a determined orientation of the interventional device;
wherein said evaluation device is configured to compare the quantity of the plurality of sensors in the field of view determined by the determination device with the data structure and generate a control signal to a feedback device to provide feedback concerning the reliability level for the determined orientation.

12. The system as recited in claim 1, wherein the determination device is configured to plot the signals from the ultrasonic tracking device as a function of a beam or line number for any given frame.

13. The system as recited in claim 11, wherein:
the control signal generated by the evaluation device is sent to a graphic processing device; and
the graphic processing device is configured to generate a predetermined graphic on a display indicative of the reliability level for the determined orientation.

14. The system as recited in claim 11, wherein the control signal generated by the evaluation device is sent to an audio device and the audio device is configured to generate a predetermined audible message indicative of the reliability level in response to the control signal.

15. The system as recited in claim 11, wherein:
the evaluation device is configured to receive the determined quantity of the plurality of sensors in the field of view from the determination device and an orientation of the determination device and determine a direction to move an ultrasound transducer of the ultrasonic imaging device to provide a more reliable determination of the determined orientation; and
the evaluation device is configured to generate a second control signal to a graphic processing device to generate a graphic on a display indicating the direction to move the ultrasound transducer to provide a more reliable determination of the determined orientation.

16. A method for determining the reliability of an ultrasonic tracking device that is configured for tracking an orientation of an interventional device having a plurality of sensors, comprising the steps of:
receiving signals from the ultrasonic tracking device and determining a quantity of the plurality of sensors in a field of view of the ultrasonic tracking device;
comparing the determined quantity of the plurality of sensors to a data structure that correlates a reliability level for a determined orientation of the interventional device with the quantity of the plurality of sensors in the field of view of the ultrasonic tracking device; and
providing feedback concerning the reliability level for the determined orientation.

17. The method as recited in claim 16, wherein the signals from the ultrasonic tracking device comprise a signal stream received from a signal processor and the signals are plotted as a function of a beam or line number for any given frame.

18. The method as recited in claim 16, wherein the feedback comprises a predetermined graphic that is generated on a display indicative of the reliability level for the determined orientation.

19. The method as recited in claim 16, wherein the feedback comprises a predetermined audible message that is generated on an audio device indicative of the reliability level.

20. The method as recited in claim 16 comprising the further steps of:
determining a direction to re-position an ultrasound transducer to provide a more reliable determination of the determined orientation based on the determined quantity of the plurality of sensors in the field of view and an orientation of the determination device; and
generating a graphic on a display indicating the direction to re-position the ultrasound transducer.
